(19) United States
(12) Patent Application Publication
(10) Pub. No.: US 2010/0229274 A1
(43) Pub. Date: Sep. 16, 2010

(54) PIVOTING HEADGEAR SYSTEM

(76) Inventors: Lars-Olov Ahlgren, Fellingsbro (SE); Niklas M.S. Lilenthal, Leksand (SE); Arne O. Linderstrand, Leksand (SE)

Correspondence Address: 3M INNOVATIVE PROPERTIES COMPANY, PO BOX 33427, ST. PAUL, MN, 55133-3427, US

(21) Appl. No.: 12/681,858

(22) PCT Filed: Oct. 6, 2008
(86) PCT No.: PCT/US2008/078920

§ 371 Date: Apr. 6, 2010

Related U.S. Application Data
(60) Provisional application No. 60/978,829, filed on Oct. 10, 2007.

(51) Int. Cl. A61F 9/06; A42B 3/04

(52) U.S. Cl. 2/8.2; 2/416

(57) ABSTRACT

A pivoting protective headgear system includes a head suspension and two pivots coupled to the head suspension. A protective headgear element is pivotally connected to each pivot. In one exemplary embodiment, two transition arms are coupled to the head suspension and extend longitudinally extending away from the head suspension, and each transition arm has a distal end and a pivot at the distal end. In another exemplary embodiment, two pivots are coupled to the head suspension and each pivot has an axis of rotation configured to be below any portion of an ear of a user. In a further exemplary embodiment, two pivots are each spaced apart from the headband and coupled to the headband, and the headband is configured to be disposed against a forehead of a user and each pivot has an axis of rotation below the headband.

### FIELD

The present disclosure relates to a pivoting headgear system having a lowered axis of rotation and more particularly to a protective pivoting headgear system having a lowered axis of rotation.

### BACKGROUND

Protective headgear, for example construction hard hats, helmets, such as fire fighter helmets, shields such as welding shields and grinding shields, sports equipment headgear, and the like often include a headgear support or head suspension. The headgear supports or head suspension often include headbands and/or head straps that are adjustable so that the headgear can be supported comfortably on any size head by the headband.

In many headgear supports or head suspensions, it is desired that the headband be easy to adjust and that it fit adequately without discomfort to the user. In some cases, it is also desired that headgear supports or head suspension headbands do not require that the headgear be removed from the user's head in order to make adjustments in the size of the head suspension. Furthermore, it is desired that the head suspension be comfortable to wear for extended periods of time without excessive need of re-positioning or adjustments. Accordingly, there is a need for head suspensions that are more comfortable to wear for longer periods of time and that are easier to adjust.

### BRIEF SUMMARY

The present disclosure relates to a pivoting headgear system having a lowered axis of rotation and more particularly to a protective pivoting headgear system having a lowered axis of rotation.

In a first embodiment, a pivoting protective headgear system includes a head suspension and two transition arms. Each transition arm is coupled to the head suspension and longitudinally extending away from the head suspension. Each transition arm has a distal end and a pivot at the distal end. A protective headgear element is pivotally connected to each pivot.

In another embodiment, a pivoting protective headgear system includes a head suspension and two pivots coupled to the head suspension. Each pivot has an axis of rotation configured to be below any portion of an ear of a user. A protective headgear element pivotally connected to each pivot.

In a further embodiment, a pivoting protective headgear system includes a head suspension having a laterally extending headband and two pivots. Each pivot is spaced apart from the headband and coupled to the headband. The headband is configured to be disposed against a forehead of a user and each pivot has an axis of rotation below the headband. A protective headgear element is pivotally connected to each pivot.

### BRIEF DESCRIPTION OF THE DRAWINGS

The invention may be more completely understood in consideration of the following detailed description of various embodiments of the invention in connection with the accompanying drawings, in which:

FIG. 1 illustrates schematic side views of a traditional welding shield in a raised position, an intermediate position and a lowered position;

FIG. 2 illustrates schematic side views of an illustrative pivoting headgear according to the present disclosure in a raised position, an intermediate position, and a lowered position and;

FIG. 3 is a schematic side view of an illustrative head suspension for a pivoting headgear shown in FIG. 2; and

FIG. 4 is a schematic front view of an illustrative head suspension for a pivoting headgear shown in FIG. 2.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

In the following description, reference is made to the accompanying drawings that form a part hereof, and in which are shown by way of illustration several specific embodiments. It is to be understood that other embodiments are contemplated and may be made without departing from the scope or spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

The present disclosure relates to a pivoting headgear system having a lowered axis of rotation and more particularly to a protective pivoting headgear system having a lowered axis of rotation. This disclosure provides a lowered headgear (e.g., welding helmet or shield, hard hat fire fighter helmets, grinding shields, sports equipment headgear, and the like) pivot axis of rotation of the head suspension, as compared to other designs. This may result in a better-balanced and consequently more comfortable headgear in fixed positions and also during raising and lowering (i.e., pivoting) of the headgear. By lowering the pivot axis of rotation, the headgear and head protection element can operate closer to the user's head and provide for easier maneuvering in narrow spaces. The lower positioned pivot axis of rotation also can lower the adjustment force required to pivot the headgear, by moving the center of gravity of the pivoting headgear element toward the rotation center. While the present invention is not so limited, an appreciation of various aspects of the invention will be gained through a discussion of the examples provided below.

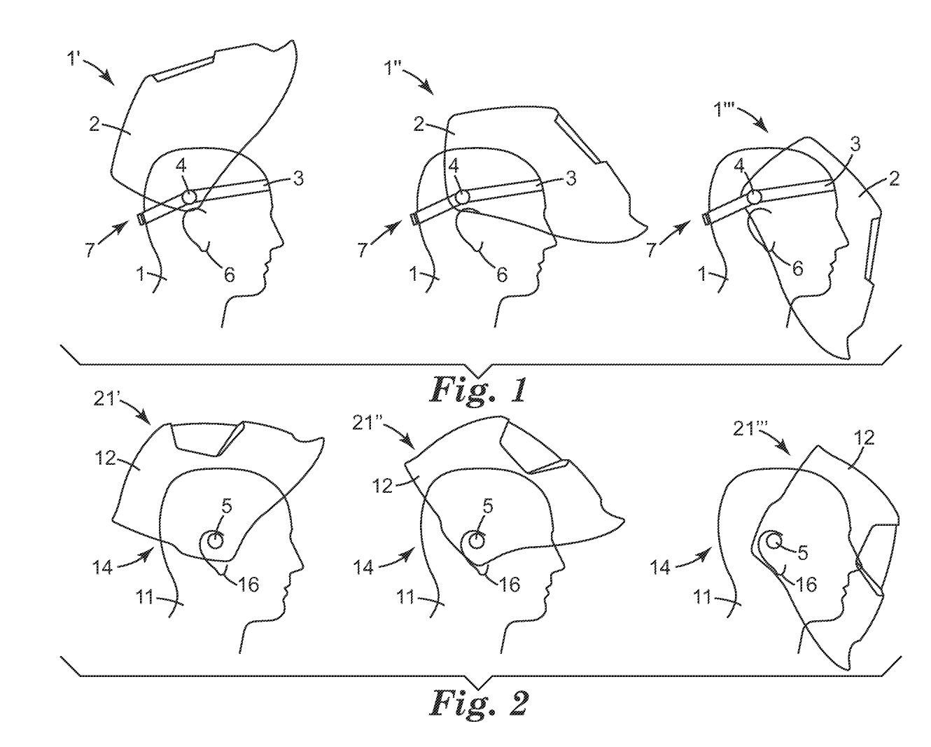

FIG. 1 illustrates schematic side views of a traditional welding shield system 7, which shows a welding shield 2 in a raised position 1′, a lowered position 1′″, and an intermediate position 1″ on a user 1. Many current designs provide a head suspension headband 3 with a pivot 4 attached on the head suspension headband 3, as illustrated. The pivot 4 provides the axis of rotation allowing the welding shield 2 to pivot between the raised position 1′ and lowered position 1′″. These designs feature the pivot 4 axis of rotation located above the user's ear 6 and/or in line with the head suspension headband 3, as illustrated.

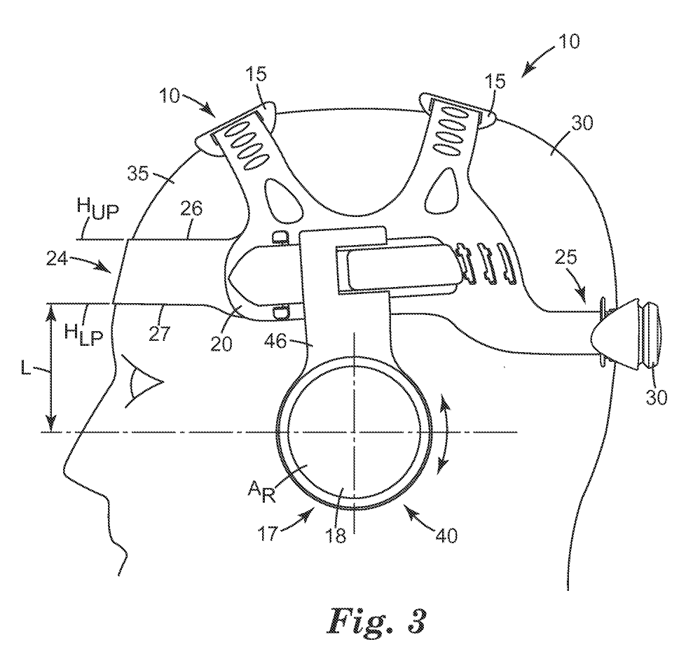

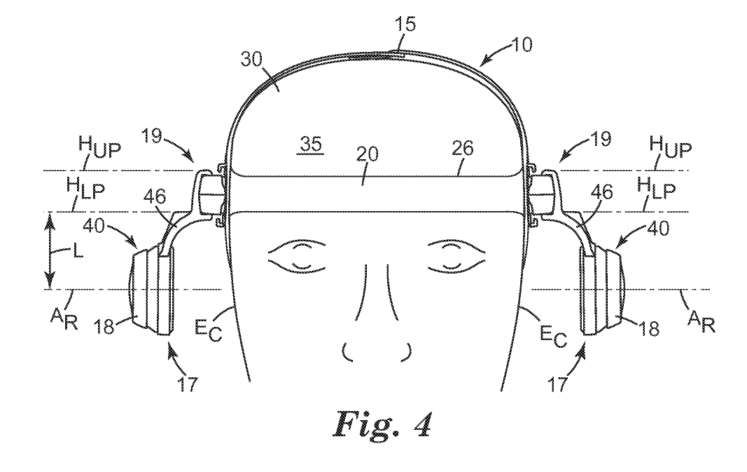

FIG. 2 illustrates schematic side views of an illustrative pivoting headgear system 14 which may be a pivoting protective headgear system. The headgear element 12 (which may be a protective headgear element such as a welding helmet or shield, hard hat fire fighter helmets, grinding shields, sports equipment headgear, and the like) of the illustrative pivoting headgear system 14 is shown in a raised position 21′, a lowered position 21′″, and an intermediate position 21″ on a user 11. This disclosure provides a head suspension headband (shown in more detail in FIG. 3 and FIG. 4) with a pivot 5 placed lower on the user's 11 head than the traditional pivot shown in FIG. 1. The pivot 5 provides the axis of rotation allowing the headgear 12 to pivot between the raised position 21′ and lowered position 21′″. In some embodiments, these designs feature the pivot 5 axis of rotation located at or below any portion of the user's ear 16 and/or below the head suspension headband, as illustrated. By lowering the pivot 5 axis of rotation, the head protection element 12 can operate closer to the head 11 and provide for easier maneuvering in narrow spaces. The low positioned pivot 5 axis of rotation can lower the adjustment force required to pivot the head protection element 12, by moving the center of gravity toward the rotation center.

FIG. 3 is a schematic side view of an illustrative head suspension for a pivoting headgear shown in FIG. 2. FIG. 4 is a schematic front view of an illustrative head suspension for a pivoting headgear shown in FIG. 2.

The head suspension 10 includes a headband 20 that extends about a user's head 30. The head suspension 10 includes one or more top bands 15 that extend over the user's head 30 and each end of the top band is joined to the headband 20. The head suspension 10 includes a head protection attachment element 40.

The head protection attachment element 40 couples a head protection element 12 (see FIG. 2) to the head suspension 10. In many embodiments, the head protection attachment element 40 pivotally couples the head protection element 12 to the head suspension 10 allowing the head protection element 12 to pivot relative to the head suspension 10. The head protection element 12 can be any useful head protection element such as, for example, a welding helmet or shield, a hard hat, a fire fighter helmet, a grinding shield, sports equipment headgear, and the like. Protective headgear may offer various types and combinations of protection, such as eye protection, head protection, etc.

The attachment element 40 can be any useful element that pivotally couples the head protection element 12 to the head suspension 10 allowing the head protection element 2 to pivot relative to the head suspension 10. Illustrative attachment elements 40 include a fastener that can adjust the frictional engagement between the pivoting head protection element 12 and the attachment element 40.

Two transition arms 46 are attached to the head suspension 10, and in many embodiments, to or at a headband 20 portion of the head suspension 10. The transition arms 46 are coupled to the head suspension 10 and longitudinally extend away from the head suspension 10, as illustrated. Each transition arm 46 is coupled to the head suspension at a proximal end 19 of the transition arm 46 and an opposing distal end 17 of the transition arm 46 includes a pivot 18. The head protection element 12 is pivotally connected to each pivot 18. The transition arms 46 can take any useful form other than the illustrated form.

The headband 20 is an elongated laterally extending element having a band length and includes a front portion 24 and a rear portion 25. The headband 20, which generally may have any useful shape, has an upper edge 26 and an opposing lower edge 27, which may or may not be straight and/or continuous. The headband 20 upper edge 26 defines an upper surface plane H_{UP}. The headband 20 lower edge 27 defines a lower surface plane H_{LP}. The headband 20 defines a range of lateral planes between the upper surface plane H_{UP} and the lower surface plane H_{LP}. The headband 20 front portion 24 is configured to be disposed against a forehead 35 of the user.

Each transition arm 46 longitudinally extends away from the headband 20 any useful length. Each transition arm 46 can have any useful length L defined by a linear distance between the headband 20 lower surface plane H_{LP} to the pivot 18 axis of rotation A_{R}. This length L can be, for example, in a range from 2 centimeters or greater, or 3 centimeters or greater, or in a range from 2 to 10 centimeters, or in a range from 3 to 6 centimeters.

In many embodiments, any portion of the transition arm 46 (such as a distal end of the transition arm 46) extends over and past (i.e., below) any portion of an ear of the user. For example, any portion of the transition arm 46 (such as a distal end of the transition arm 46) may extend below the top edge of an ear of the user. In some embodiments, any portion of the transition arm 46 (such as a distal end of the transition arm 46) extends over and past (i.e., below) an ear canal E_{C} (i.e., external acoustic meatus) of the user.

The headband rear portion 25 can include an adjustment element 50 configured to adjust a head size of the head suspension 10 with respect to a user's head. One exemplary adjustment element 50 is an adjustment knob providing a ratchet-type head size adjustment as described in U.S. Pat. No. 4,942,628, and incorporated by reference herein to the extent is does not conflict with the present disclosure. However, any other types of adjustments that enable a user to tighten and/or loosen the head suspension 10 about a users head are within the scope of the present disclosure.

Each pivot 18 is longitudinally spaced apart from the head suspension 10 or headband 20 any useful length, L as described above. In many embodiments, the pivot 18 is spaced apart from the headband 20 a length of in a range from 2 centimeters or greater, or 3 centimeters or greater, or in a range from 2 to 10 centimeters, or in a range from 3 to 6 centimeters. In many embodiments, the pivot 18 extends over or past (i.e., below) any portion of an ear of the user. For example, the pivot 18 may be disposed below the top edge of an ear of the user. In some embodiments, the pivot 18 extends over or past (i.e., below) an ear canal E_{C} (i.e., external acoustic meatus) of the user.

Each pivot 18 has an axis of rotation A_{R}. The axis of rotation A_{R} is longitudinally spaced apart from the head suspension 10 or headband 20 by any useful length. In many embodiments, the axis of rotation A_{R} is spaced apart from the headband 20 a length of in a range from 2 centimeters or greater, or 3 centimeters or greater, or in a range from 2 to 10 centimeters, or in a range from 3 to 6 centimeters. In many embodiments, the axis of rotation A_{R} is spaced apart from the headband 20 lateral plane a length of in a range from 2 centimeters or greater, or 3 centimeters or greater, or in a range from 2 to 10 centimeters, or in a range from 3 to 6 centimeters. In some embodiments, the axis of rotation A_{R} is spaced apart from the headband 20 lower surface 27 plane H_{LP} a length L in a range from 2 centimeters or greater, or 3 centimeters or greater, or in a range from 2 to 10 centimeters, or in a range from 3 to 6 centimeters. In many embodiments, the axis of rotation A_{R} extends over and past (i.e., below) any portion of an ear of the user. In some embodiments, the axis of rotation A_{R} extends over or past (i.e., below) an ear canal E_{C} (i.e., external acoustic meatus) of the user. In some embodiments, the axis of rotation A_{R} is within a radius of 30 millimeters, or within a radius of 3 centimeters, or within a radius of 2 centimeters, or within a radius of 1 centimeter of the ear canal of a user.

The lowered pivot axis of rotation, described above can result in better-balanced head protection element in fixed positions and also during raising and lowering (i.e., pivoting) of the head protection element. By moving the center of gravity of the pivoting head protection element toward the rotation center, the lowered pivot axis of rotation can reduce the adjustment force required to pivot the headgear and reduce stresses on the user's head during use and during raising and lowering (i.e., pivoting) of the head protection element.

Thus, embodiments of the PIVOTING HEADGEAR SYSTEM are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A pivoting protective headgear system comprising:
a head suspension;
two transition arms, each transition arm coupled to the head suspension and longitudinally extending away from the head suspension, and each transition arm having a distal end and a pivot at the distal end; and
a protective headgear element pivotally connected to each pivot.

2. A pivoting protective headgear system according to claim 1, wherein the head suspension comprises a laterally extending headband and each transition arm coupled to the head band.

3. A pivoting protective headgear system according to claim 1, wherein the protective headgear element is a welding helmet.

4. A pivoting protective headgear system according to claim 1, wherein each pivot has an axis of rotation that is configured to be below any portion of an ear of a user.

5. A pivoting protective headgear system according to claim 2, wherein the headband portion is configured to be disposed against a forehead of a user and each pivot has an axis of rotation that is configured to be below any portion of an ear of a user.

6. A pivoting protective headgear system according to claim 1, wherein each transition arm has a length being 20 millimeters or greater.

7. A pivoting protective headgear system comprising:
a head suspension;
two pivots coupled to the head suspension and each pivot has an axis of rotation configured to be below any portion of an ear of a user; and
a protective headgear element pivotally connected to each pivot.

8. A pivoting protective headgear system according to claim 7, wherein the head suspension comprises a laterally extending headband and each pivot is longitudinally spaced apart from the headband.

9. A pivoting protective headgear system according to claim 7, wherein the head protection element is a welding helmet.

10. A pivoting protective headgear system according to claim 7, wherein each pivot has an axis of rotation that is a radius of 25 millimeters of an ear canal of a user.

11. A pivoting protective headgear system according to claim 8, further comprising two transition arms, each transition arm coupled to the headband and longitudinally extending away from the headband, and each transition arm having a distal end and the pivot at the distal end.

12. A pivoting protective headgear system according to claim 11, wherein each transition arm has a length being 20 millimeters or greater.

13. A pivoting protective headgear system comprising:
a head suspension having a laterally extending headband;
two pivots, each pivot being spaced apart from the headband and coupled to the headband, and the headband being configured to be disposed against a forehead of a user and each pivot has an axis of rotation below the headband; and
a protective headgear element pivotally connected to each pivot.

14. A pivoting protective headgear system according to claim 13, further comprising two transition arms, each transition arm coupled to the headband and longitudinally extending away from the headband, and each transition arm having a distal end and the pivot at the distal end.

15. A pivoting protective headgear system according to claim 14, wherein headband defines a lateral plane and each pivot has an axis of rotation to be below the lateral plane.

16. A pivoting protective headgear system according to claim 14, wherein headband has an upper surface and an opposing lower surface and the lower surface defines a lateral plane and each pivot has an axis of rotation to be below the lateral plane.

17. A pivoting protective headgear system according to claim 13, wherein the protective headgear element is a welding helmet.

18. A pivoting protective headgear system according to claim 13, further comprising two transition arms, each transition arm fixed to the headband portion and longitudinally extending away from the headband portion length, and each transition arm having a distal end and the pivot at the distal end.

19. A pivoting protective headgear system according to claim 13, wherein the headband portion is configured to be disposed against a forehead of a user and each pivot has an axis of rotation is configured to be adjacent to an ear of a user.

20. A pivoting protective headgear system according to claim 18, wherein the transition arms have a length of 20 millimeters or greater.
